(19) United States
(12) Patent Application Publication
(10) Pub. No.: US 2006/0089701 A1
(43) Pub. Date: Apr. 27, 2006

(54) In situ molded stent and method and system for delivery

(75) Inventors: Stephen David Forsyth, Ottawa (CA); Max R. Wood, Cantley (CA); Kemal Schankereli, Stillwater, MN (US); Kenneth G. Thurston, West Grove, PA (US)

Correspondence Address: PEARNE & GORDON LLP, 1801 EAST 9TH STREET, SUITE 1200, CLEVELAND, OH, 44114-3108, US

(73) Assignee: Matrix Medical, LLC, Palo Alto, CA (US)

(21) Appl. No.: 10/973,747

(22) Filed: Oct. 26, 2004

(51) Int. Cl. A61F 2/06

(52) U.S. Cl. 623/1.11

(57) ABSTRACT

A method for forming a stent in situ involves manipulating a delivery system to provide a mold within a lumen of a living body, and injecting a settable, biocompatible phase invertible composition into the mold. After the biocompatible phase invertible composition is set, the delivery system is removed. The stent provides a micro-porous support structure with good tensile strength that is adhesively bound to the lumen. The biocompatible phase invertible composition may be a composition concocted from albumin and collagen, for example, and may be infused with an anti-restenosis agent.

### CROSS-REFERENCE TO RELATED APPLICATIONS

This is the first application filed for the present invention.

### MICROFICHE APPENDIX

Not Applicable.

### TECHNICAL FIELD

The present invention relates in general to structural body implants, and in particular to a stent that is molded in situ to support a lumen in a living body.

### BACKGROUND OF THE INVENTION

According to the Department of Health and Human Services, in 2002, 12.3% of the population of the United States was at least 65 years old, and that this fraction of the population is expected to rise to 20% by 2030. With a growing seniors population, the prevalence of many diseases, including those that require the unblocking of occluded lumens (such as blood vessels, ducts, ureters etc.) within the body, increases as well.

For example, it is known to implant a stent to support a lumen while healing takes place, or to keep a body lumen such as a duct, ureter or blood vessel open. Typically stents are formed from stainless steel, which has the requisite tensile strength to apply an expansive force when implanted. Since in many cases the stents are designed to be permanently implanted in a patient, it is necessary to ensure that the walls of the supported tissue continues to be nourished, etc. Accordingly, stents are typically formed of a metal mesh or coil structure, or the like.

In order for the stent to be securely retained at the implant site, a predetermined force must be applied to the wall of the body lumen. In an angioplasty operation, both the angioplasty and the stent traumatize the wall of the artery, resulting in smooth muscle growth etc., which tend to promote subsequent occlusion of the artery, i.e. restenosis. In accordance with the prior art, it is also known to apply a coating to the stent to reduce the likelihood of rejection and/or to provide a controlled delivery of a drug that inhibits smooth muscle growth. Such stents are known as drug eluting stents. While the use of drug eluting stents significantly reduces restenosis in the short term, because a supply of the drug is limited, in the longer term, restenosis may occur. While drug eluting stents are still relatively new technology, there are indications that long term restenosis rates may not be significantly improved.

While prior art stents are useful, the complexities involved in implanting such stents is considerable. There are several methods for compacting such devices for insertion into a catheter, and delivery along a torturous path to the implant site. The various methods include pre-forming the stent by application of a heat treatment, collapsing the stent to fit a mold, and then inserting the stent into the catheter. Certain mesh structures for stents are also conducive to axial elongation to reduce the diameter of the stent. In any case the strength of the stent and its ability to elastically deform (i.e. to return to a desired configuration after having been compressed), which is essential to the function of the stent, restricts the design options and increases the weight of the stent.

Furthermore, the ability for the stent to assume a shape and configuration of the wall of a treated artery is limited. There are a finite number of sizes of stents that are available, and the requirement that the stent be immobilized requires the selection of a stent that applies adequate force on the wall of the artery. Any fit that is less than ideal can accelerate restenosis, and/or decrease stability of the stent.

Accordingly, there exists a need for improved stents that can be delivered and implanted more efficiently, and inhibit restenosis more effectively. There is also a requirement for stents that can more effectively conform to tissue to the supported, and that do not rely on drug elution to inhibit restenosis.

### SUMMARY OF THE INVENTION

It is therefore an object of the invention to provide a stent that reduces the probability of restenosis by reducing the trauma to the tissue to be supported.

It is also an object of the invention to provide a stent that readily conforms to the tissue to be supported and provides a uniform, distributed support without pressure points or pressure voids.

Another object of the invention is to provide a method and delivery system for implanting a stent in situ within a body.

In accordance with an aspect of the invention, an apparatus for molding a stent at a selected site within a lumen of a living body, is provided. The apparatus includes a catheter having a distal insertion end, and a proximal manipulation end. On the distal insertion end a distal end unit is provided, the distal insertion end being positioned within the lumen by maneuvering the proximal manipulation end of the catheter. The distal end unit incorporates a mandrel that is expandable from a collapsed insertion condition to an expanded molding condition in which a mold space is defined between a wall of the lumen and the mandrel.

A conduit of the catheter is provided for injecting a biocompatible phase invertible composition into the mold space to fill the mold space. The biocompatible phase invertible composition sets to form a rigid, micro-porous stent that provides structural support for the lumen.

A method of molding a stent for supporting a lumen at a site in a living body is also provided. The method involves operating a stent delivery system to position a mandrel within the lumen at the site, operating the stent delivery system to define a mold space between the mandrel and the lumen, and injecting a biocompatible phase invertible composition into the mold space to fill the mold space, the biocompatible phase invertible composition setting to form a micro-porous stent that provides structural support for the lumen.

Another aspect of the invention is a stent formed in situ within a living body, the stent being made of a biocompatible phase invertible composition molded to form the stent, the biocompatible phase invertible composition being selected so that it sets in situ to form a rigid, micro-porous stent that provides structural support for a lumen in the living body.

### BRIEF DESCRIPTION OF THE DRAWINGS

Further features and advantages of the present invention will become apparent from the following detailed description, taken in combination with the appended drawings, in which:

FIGS. 1a, 1b, 1c and 1d are schematic illustrations of four respective operative states of a stent delivery system in accordance with an embodiment of the invention;

FIG. 2 is a schematic illustration of a cross-section of a catheter in accordance with one embodiment of the invention;

FIGS. 3a and 3b are schematic illustrations of a mandrel of the stent delivery system shown in FIGS. 1a, 1b, 1c and 1d in two respective operative states; and

FIGS. 4a, 4b, 4c, 4d, 4e, 4f and 4g are schematic illustrations of six stages in the implantation of a stent using the delivery system shown in FIGS. 1a, 1b, 1c and 1d.

It should be noted that throughout the appended drawings, like features are identified by like reference numerals.

### DETAILED DESCRIPTION OF THE PREFERRED EMBODIMENT

The invention provides a method and delivery system for forming a stent molded in situ in a lumen of a living body. Because the stent is molded in situ, it conforms ideally with a wall of the lumen. Consequently, the stent provides uniform support for the wall of the lumen, without pressure points or pressure voids. Furthermore, a biocompatible phase invertible composition used to mold the stent is a bio-adhesive. Consequently, the stent binds to the lumen wall, but remains porous to permit nutrients and other vital substances required by the lumen wall to be transported across the stent. The biocompatible phase invertible composition provides a stent having a tensile strength required to provide structural support for the lumen, and having a smooth inner surface that does not interfere with bodily fluid circulation.

One example of a biocompatible phase invertible composition suitable for use in molding a stent in situ is a biocompatible phase invertible composition developed by an inventor of the instant invention, and described in detail in co-pending U.S. patent application Ser. No. 10/635,847, filed on Aug. 5, 2003, the specification of which is incorporated herein by reference in its entirety. The biocompatible phase invertible composition includes an aldehyde modified to be biocompatible, with albumin and collagen.

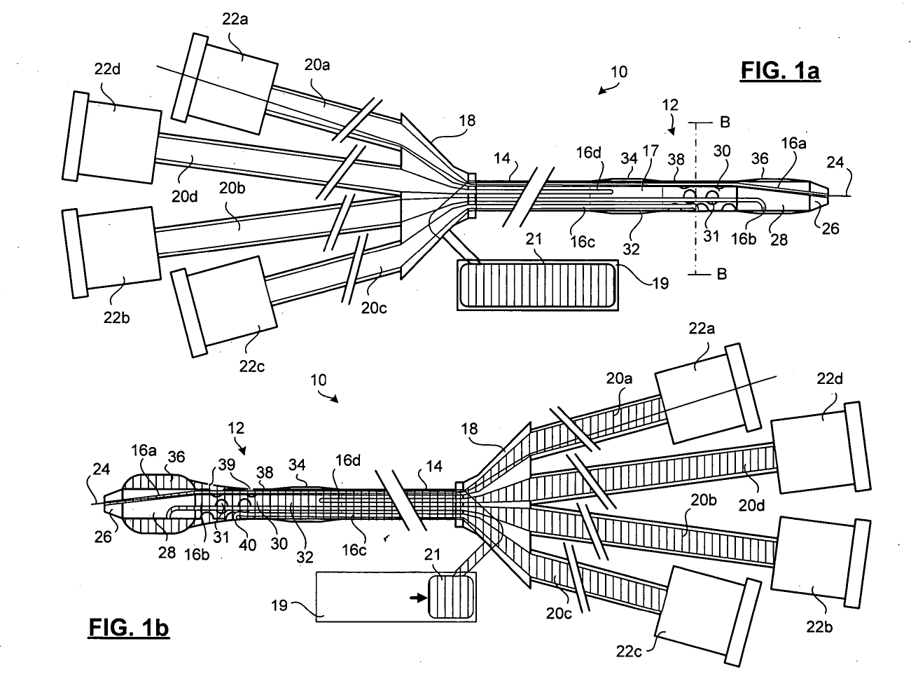

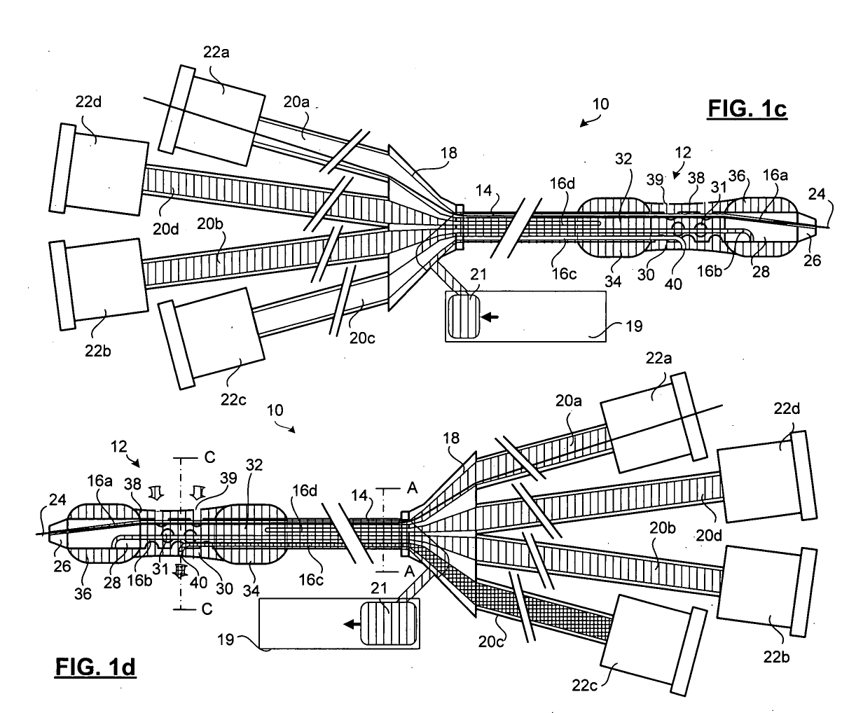

FIGS. 1a, 1b, 1c and 1d are schematic illustrations of an embodiment of a stent delivery system 10 for molding the stent in accordance with the invention. The apparatus is shown in four states of operation in the respective figures.

In FIG. 1a, the stent delivery system 10 is shown in a state in which a distal end unit 12 can be moved within a lumen of a body. The distal end unit 12 is connected to a catheter at its distal, insertion end, and movement of the distal end unit 12 is effected by operating a proximal manipulation end of the catheter.

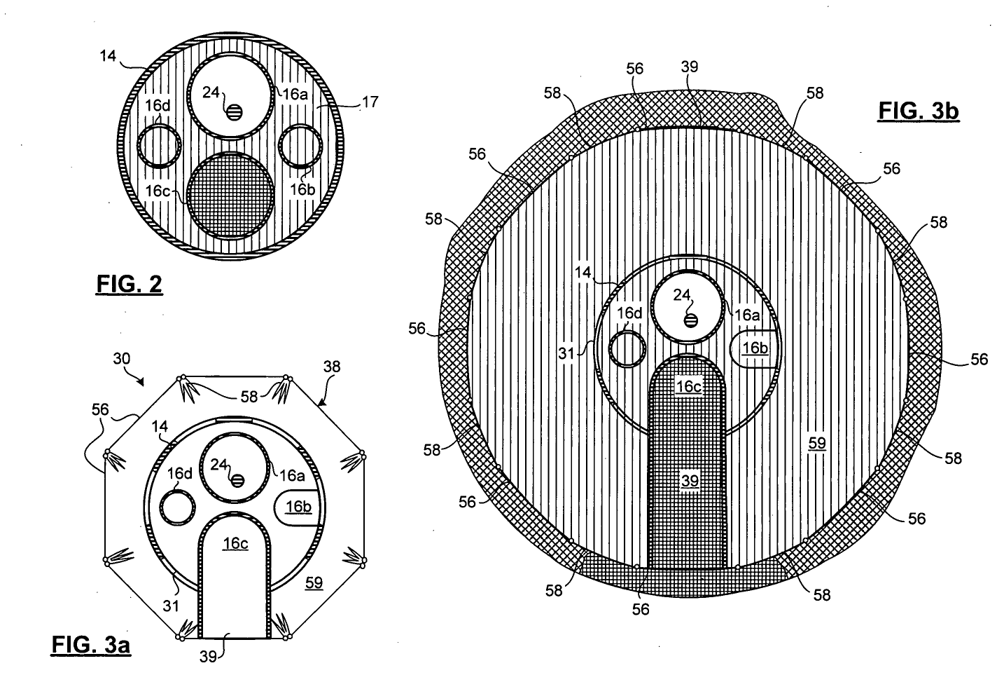

The schematically illustrated catheter of the embodiment shown in FIGS. 1a, 1b, 1c and 1d is a multi-lumen catheter 14, which encases four micro-tubes 16. To facilitate visual association of the respective micro-tubes 16a, 16b, 16c and 16d with corresponding components of the distal end unit 12, the micro-tubes 16a, 16b, 16c and 16d are shown as if they were all of equal cross-sectional area, and disposed in a common plane. This is for purposes of illustration only. One embodiment of the multi-lumen catheter 14 is more accurately, though schematically, depicted in a cross-sectional view shown in FIGS. 2, 3a and 3b.

The multi-lumen catheter 14 is coupled to a hub 18, which serves to interconnect respective micro-tubes 16a, 16b, 16c and 16d with corresponding connectors 20a, 20b, 20c and 20d. Each of the connectors 20a, 20b, 20c and 20d has an adapter end 22a, 22b, 22c, 22d, respectively, which facilitates sealed connection to a pressurized fluid controller, or the like. The adapter ends 22a, 22b, 22c, 22d may be Luer locks, well known in the art, for example. Each of the connectors 20a, 20b, 20c and 20d are coupled to a respective port of the hub 18 that is in fluid communication (within the hub 18) with a respective one of the micro-tubes 16a, 16b, 16c and 16d, so that fluid communication can be established between each micro-tube 16 and the respective connector 20.

In the multi-lumen catheter 14 spaces between the outer walls of the micro-tubes 16 and an inner wall of the multi-lumen catheter 14 provides a return channel 17 which is in fluid communication with a port of the hub 18, that is connected to a fluid reservoir 19. The fluid reservoir 19 includes an elastic bladder 21, for example, that can be contracted or expanded to inject or remove fluid from the return channel 17. The fluid reservoir 19 may also be used to supply fluid to the micro-tubes 16 via the respective adapters 22, in a manner well known in the art.

As is well known in the art, a guide wire 24 is typically inserted into the lumen and directed through the lumen to a position beyond the site where the stent(s) are to be implanted. Once a distal end of the guide wire 24 is in place, the proximal end of the guide wire 24 is threaded through the stent delivery system 10 starting at the distal end unit 12. The guide wire 24 is passed through the tube 16a, the hub 18, and the connector 20a to emerge through the adapter end 22a. The multi-lumen catheter 14 is then slid over the guide wire 24 until the distal end unit 12 reaches the site.

The distal end unit 12 has three principal components, a distal balloon section 28 (with a distal tip 26), a mandrel-forming midsection 30, and a proximal balloon section 32. The mandrel-forming midsection 30 and distal balloon section 28 are separated by an end wall that has openings for sealably retaining the micro-tubes 16a, 16b that pass through the end wall, in accordance with the illustrated embodiment. The distal tip 26 may further have a wall for sealed passage of the tube 16a, or for the guide wire 24. The sealed off space within the distal balloon section 28 contains a fluid, such as air or a biocompatible heparin/saline solution well known in the art.

Technologies associated with balloons for stretching lumens are well developed. There are many different types of balloons with respective properties associated with inflation, an ability to fold and collapse to a minimum profile, durability, etc. There are also many different inflation mechanisms for such balloons, including remotely activated balloons, etc., any of which may be applied to embodiments of the present invention. In the illustrated embodiment, respective balloons of the proximal balloon section 32 and the distal balloon section 28 are coupled to ends of corresponding micro-tubes 16b and 16d, and so control of fluid pressure within the micro-tubes 16b, 16d results in control of a diameter of the respective proximal and distal balloons. As is shown in profile, tube 16b passes radially through the outer wall of the multi-lumen catheter 14 and into sealed fluid communication with the distal balloon to permit flow of a fluid that can be safely injected into the lumen (such as heparin/saline solution, if the lumen is a blood vessel). Similarly the tube 16d is coupled to the proximal balloon.

The outer wall of the multi-lumen catheter 14 is a solid fluid-retaining wall that extends between the hub 18 and a distal tip 26 of the distal end unit 12, with the exception that within the mandrel-forming midsection 30, the outer wall is fluid-permeable. As shown in the illustrated embodiment, fluid passages 31 are provided through the outer wall within the mandrel-forming midsection 30 that permit fluid communication between the channel 17 and a mold space between a flexible mandrel wall 38 and the lumen.

While the foregoing is one example of a distal end unit 12 for delivering a mandrel to an implant site to enable the in situ molding of a stent, other configurations are also contemplated. For example, a different number of balloons may be used, each balloon having an individual or shared inflation system, and the mandrel wall can be formed by any combination of parts of the balloons or other surfaces of the distal end unit to provide a mold for the stent, and for permitting injection of a biocompatible phase invertible composition into the mold.

As will be apparent to those skilled in the art, the stent delivery system 10 as shown in FIG. 1a is prepared for insertion into a living body by filling the delivery system with a biocompatible fluid that may be safely injected into the lumen. Each of the micro-tubes 16 is purged to remove all air before the multi-lumen catheter 14 enters the lumen, as shown in FIG. 1b.

As is well known in the art, inflation of the proximal and distal balloons is usually performed by supplying a biocompatible fluid to the balloon, the fluid being chosen so that if a balloon ruptures or leaks, no adverse consequences result. In the illustrated embodiment, the fluid supply is provided through the micro-tubes 16b, 16d which are in fluid communication with the proximal balloon section 28 and the distal balloon section 32. In the case of an arterial transluminal angioplasty, the fluid may be air or a heparin/saline solution, as noted above.

In FIG. 1b, the distal balloon is inflated by increasing a pressure of the fluid within the micro-tube 16b, the corresponding path within the hub 18, and within connector 20b. This inflation may be designed to permit dilatation of the lumen to varying degrees, for defining a distal end of a mold space formed between the wall of the lumen and the mandrel wall 38, and, for the expansion of the mandrel wall 38. The flexible mandrel wall 38 is designed to radially expand/contract when either of the proximal and distal balloons are inflated/deflated. The concurrent expansion and contraction of the mandrel wall 38 is accomplished by the communication of tensile forces from the balloons to which the mandrel wall 34 is attached, as shown, but may also be accomplished by one or more separate actuators, for example. The mandrel wall 34 with the proximal and distal balloons defines the interior wall of a mold, as will be explained below in more detail.

Because the space between the mandrel wall 38 and the outside of the outer wall of the multi-lumen catheter 14 is in fluid communication with the channel 17 (by virtue of fluid passages 31), when the mandrel wall 38 is radially expanded by the expansion of the distal balloon, a pressure drop in the fluid within the channel 17 draws fluid from the bladder 21. Similarly, expansion of the proximal balloon by increasing a pressure in tube 16d draws fluid from the bladder 21.

The micro-tube 16c is routed within the multi-lumen catheter 14 so that a distal end of the micro-tube 16c passes through one of the fluid passages 31, and passes through the mandrel wall 38 to provide an ejection nozzle 40 for delivering the biocompatible phase invertible composition, as is further described below. The nozzle 40 is secured to the flexible mandrel wall 38. The micro-tube 16c is flexible and moves with the radial motion of the mandrel wall 38. The mandrel wall 38 further includes one or more apertures 39 which are preferably positioned at a location that is radially opposite the nozzle 40, so that the biocompatible phase invertible composition completely fills the mold space between the outer surface of the mandrel and the lumen wall before entering the apertures 39. One embodiment of the mandrel wall 38 is further described below with reference to FIGS. 3a and 3b.

In accordance with some applications of the stent delivery system 10, the distal end unit 12 may be used to perform an initial dilatation of the lumen before the stent is molded. Accordingly, the distal end unit 12 may be positioned first so that the proximal balloon is centered on the lumen site, and after the proximal balloon is expanded to perform an initial dilatation of the lumen, the distal end unit 12 is retracted to center the mandrel wall 38 on the lumen site. There are numerous other possible inflation sequences that may be used, as will be appreciated by those skilled in the art. For example, in an angioplasty intervention primary lumen dilatation with a standard angioplasty balloon may be required prior to stent formation. Current wisdom in the coronary intervention literature identifies minimum lumen diameter (MLD) as a potent predictor of late lumen loss and subsequent restenosis of the stented vessel. An optimal result involves achieving a maximum MLD with a smooth, consistent diameter to minimize the impact of late lumen loss, and restenosis.

As shown in FIGS. 1c and 1d, when both the proximal and distal balloons are inflated, the mandrel wall 38 is radially expanded, and the stent mold space is defined. In the extended position, the mandrel wall 38 may be slightly tapered (the degree to which the mandrel wall 38 is tapered is exaggerated for illustration) toward the distal end to facilitate removal of the distal end unit 12 from the molded stent, as will be described below in more detail.

As shown in FIG. 1d, after the mandrel wall 38 is expanded to define the mold space, the biocompatible phase invertible composition is ejected through the ejection nozzle 40. It will be appreciated that one or more nozzles of various sizes may be used for the same purpose.

The initial content of the micro-tube 16c, as shown in FIGS. 1b and 1c is a biocompatible fluid, such as a heparin/saline solution, as shown in FIG. 1d because the biocompatible phase invertible composition is a relatively fast-setting composition that must be delivered to the lumen site before it sets. That fluid is pushed ahead of the biocompatible phase invertible composition in the micro-tube 16c and is forced through the ejection nozzle 40 into the mold space. The fluid circulates around the mandrel wall 38 within the mold space, and is ejected from the mold space through the apertures 39 by a pressure difference between the fluid in the tube 16c and the channel 17. As will be appreciated by those skilled in the art, the pressure difference may be regulated by controlling a pump that injects the biocompatible phase invertible composition through the micro-tube 16c, and/or control of the elastic bladder 21. By controlling both, the mold space can pressurized to determine whether the balloons have a positive seal prior to injecting the biocompatible phase invertible composition into the mold space. If a predetermined threshold pressure is not maintained within the mold space, a leak between a lumen wall and a balloon is detected and the balloons are either further inflated, or the distal end unit 12 is repositioned, to ensure that the inflated proximal and distal balloons securely seal the mold space between the mandrel wall 38 and the wall of the lumen.

When the biocompatible phase invertible composition reaches the mold space, it circulates around the mandrel wall until it has filled the mold space and displaced the biocompatible fluid, which being less viscous is readily displaced and not inclined to mix with the biocompatible phase invertible composition.

In order to control the amount of biocompatible phase invertible composition that is forced through the apertures 39, a pre-computed volume of the biocompatible phase invertible composition is injected. In accordance with one embodiment, a chaser fluid having a higher viscosity than that of the biocompatible phase invertible composition is used to deliver the composition into the mold space. A biocompatible glycerol can be used, for example, as a chaser fluid for pushing the biocompatible phase invertible composition through the micro-tube 16 and into the mold space. A supply of the chaser fluid can be controlled to substantially clear the ejection nozzle 40 of biocompatible phase invertible composition, which may facilitate separation of the mandrel from the stent after phase inversion.

As well, the apertures 39 may be readily permeable to the biocompatible fluid but not to the biocompatible phase invertible composition, in order to ensure that the biocompatible resin is retained in the mold space. A further alternative is that suitably controlled valve means are provided that effectively close the apertures 39 to inhibit the entry of the biocompatible phase invertible composition. While the biocompatible phase invertible composition is being injected, the elastic bladder 21 expands to accomodate the fluid displaced through the apertures 39 and the channel 17.

It will be noted that throughout the attached drawings, the fluid used to initially fill the multi-lumen catheter 14 is represented by vertical hatching, and the biocompatible phase invertible composition is represented by a fine-scale crosshatched pattern. A diagonal crosshatch pattern is used to represent fluid trapped within the mold space, such as blood, for example.

FIG. 2 schematically illustrates a cross-section of the multi-lumen catheter 14 taken along section AA shown in FIG. 1d. The four micro-tubes 16a, 16b, 16c and 16d are arranged in diametrically opposed pairs, such that two larger diameter micro-tubes 16a and 16c are centered on a plane that is perpendicular to a plane of the other two axial passages 16b and 16d. The micro-tubes 16 may be spaced apart by spacers distributed longitudinally along the multi-lumen catheter 14, provided that no significant obstruction of flow through the channel 17 results. As shown, the micro-tubes 16b and 16d for controlling the inflation of the proximal balloon 34 and the distal balloon 36, respectively, do not necessarily require a micro-tube 16 having a diameter as large as that used for injecting the biocompatible phase invertible composition.

It will be appreciated by those skilled in the art that the illustrated embodiment of the multi-lumen catheter 14 is one of several styles of multi-lumen catheter that could be used. There is no requirement that the cross-section of each micro-tube 16 be circular. Furthermore, alternative fluid conduits can be used. For example, a catheter having one or more sectioning walls running longitudinally through the catheter is an alternative means of dividing the catheter capacity into respective parallel fluid conduits that provide isolated fluid communication paths through the catheter.

FIGS. 3a and 3b schematically illustrate a cross-section of the mandrel-forming midsection 30 in two principal operating states: a collapsed insertion condition; and an expanded molding condition. FIG. 3a shows the mandrel-forming midsection 30 in the collapsed insertion condition, when the proximal balloon 34 and the distal balloon 36 are collapsed and the distal end unit 12 is free to reciprocate within the lumen (see section BB in FIG. 1a): and, FIG. 3b shows the mandrel in an expanded state as shown in FIG. 1d (section CC) in which the mandrel is in the expanded molding condition.

In accordance with the illustrated embodiment of the invention, the mandrel wall 38 is composed of alternating materials of two kinds: tensile strips 56, and pleated expansive bands 58 (only two of each of which are identified by reference numeral 58 in FIG. 3a for clarity of illustration). As shown, each tensile strip 56 is connected along longitudinal edges to respective expansive bands 58, and likewise longitudinal edges of the expansive bands 58 are bonded to corresponding tensile strips 56. At the ends (not shown) of the mandrel wall 38, the tensile strips 56 are in tensile attachment to the balloons, so that expansion of the balloons tensions the tensile strips 56 and causes the radial expansion of the mandrel wall 38, the radial expansion being enabled by the unfolding and stretching of the pleated expansive bands 58.

In the embodiment shown in FIG. 3b, the tensile strips 56 are marginally wider than the extended expansive bands, and are designed to bend, but not to appreciably stretch under the tension applied from the ends. The expansive bands 58 are designed to permit the definition of a wall despite the relative (azimuthal) separation of the tensile strips 56 during the expansion.

As a matter of design choice, the apertures 39 may be provided in either or both of the tensile strips 56 and the expansive bands 58, to permit the initial content of the multi-lumen catheter 14 and the mold space to be displaced by the biocompatible phase invertible composition. As explained above, the initial fluid content flows through the apertures 39 into a mandrel space 59 between the outer wall of the multi-lumen catheter 14, and an interior surface of the mandrel wall 38, and through the fluid passages 31 (only one identified in each of FIGS. 3a and 3b for clarity) into the channel 17. Some embodiments of the mandrel wall. 38 close the apertures 39 and/or fluid passages 31 when the mandrel is in a closed (contracted) condition.

The biocompatible phase invertible composition is not reactive with body fluids, it does not erode under the conditions of the fluid naturally passing through the lumen, it does not chemically interact with the fluid, or bond with components of the fluid that pass through the lumen, etc. The biocompatible phase invertible composition may be biodegradable or otherwise absorbed into the system at a controlled rate, or may be designed to provide a permanent stent. The inner surface of the stent formed by the set biocompatible phase invertible composition is smooth and resists subsequent plaque formation, but is fluid-permeable, so that the lumen wall can be nourished.

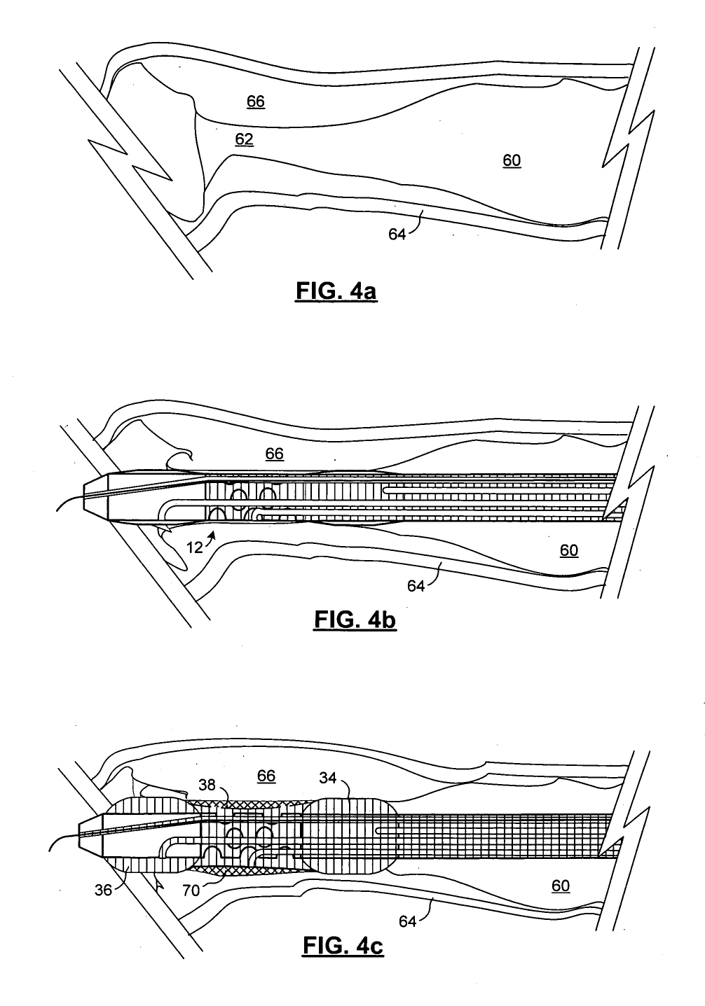

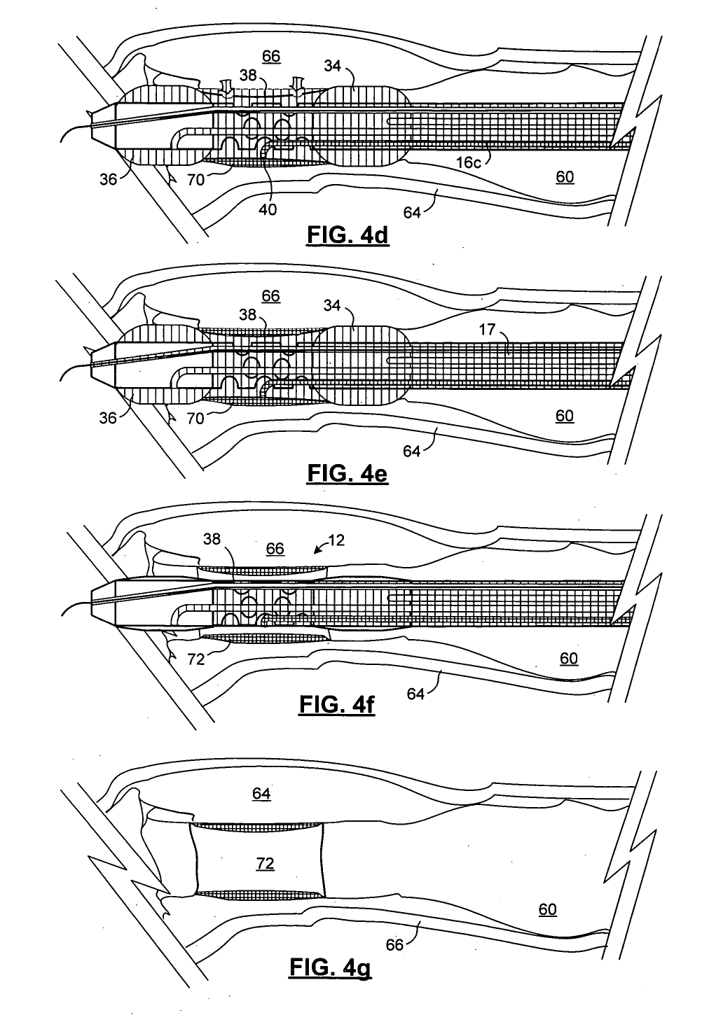

FIGS. 4a, 4b, 4c, 4d, 4e, 4f and 4g schematically illustrate seven principal steps in an exemplary method for molding a stent in accordance with an embodiment of the invention. FIG. 4a schematically illustrates a part of a lumen, which in the present embodiment is an artery 60. The artery 60 is partially occluded at a site 62. As is well known in the art, cholesterol fats deposited on a wall 64 of the artery 60 build up to form atherosclerotic plaque 66, which when sufficiently thick occludes the artery 60, resulting in arterial stenosis. This throttles blood supply to downstream tissues, and is particularly life threatening if the downstream tissues are the heart or other vital organ.

FIG. 4b shows the distal end unit 12 guided along the (previously inserted) guide wire 24, and positioned at the stent site 62, such that the distal and proximal balloon sections straddle the stent site. It will be apparent to those skilled in the art that maneuvering a catheter to bring the distal end unit 12 into position is a skill practiced by interventional teams. The surgical team may then inflate respective balloons by increasing a pressure of a fluid (such as air or a heparin/saline solution) using flow control equipment in fluid communication with the corresponding connectors 20b and 20d, in a manner well known in the art.

FIG. 4c shows the distal balloon 36 and proximal balloon 34 in expanded states. It will be appreciated by those skilled in the art that between steps shown in FIG. 4a, and FIG. 4c, the site 62 may have been subjected to a treatment for fracturing, removing, or mollifying the atherosclerotic plaque 66, for enlarging the artery, etc., and that a standard angioplasty balloon may have been used for dilatation (fracturing plaque and enlarging the artery) to achieve MDL prior to maneuvering the distal end unit 12 into position.

In some cases, the degree to which the lumen is dilated in accordance with prior art procedures can be reduced in accordance with the present invention, because the prior art stents require significant dilatation of the lumen in order to install and maintain the stent within the lumen, whereas the present invention permits the stenting force to be distributed along an extended surface area of the wall 64 ensuring localization of the stent, and further because of the active bonding of the stent material to the wall 64 less dilatation of the lumen is required. Accordingly, the stent site is exposed to less trauma, and the probability of subsequent restenosis is reduced. A damaged lumen wall created by the primary dilatation, is completely covered by the stent in accordance with the invention, thus eliminating exposure of the damaged lumen wall to thrombogenic and proliferative cellular components. In covering the damaged lumen wall with a smooth distribution (in situ molded tube, rather than lattice), point sources of stress and tissue strain are mitigated. Moreover, the micro-porous nature of the stent material permits oxygen and nutrient exchange across the stent, thus preserving underlying cellular function while minimizing exposure to potentially thrombotic elements.

With both the proximal and distal balloons expanded to contact the wall 64 surrounding the stent site 62 (or any residual/compacted plaque 66 thereon), a mold space 70 in which the stent is molded is defined. The mold space 70 is initially filled with body fluids, indicated by diagonal crosshatching.

The contact between the wall 64 and the proximal and distal balloons 34,36 is fluid tight in the illustrated embodiment. However, in other embodiments one of the proximal balloon 34 and the distal balloon 36 may be pressure regulated to permit the body fluids and the biocompatible initial fluid in the micro-tube 16c to leak past the balloon while inhibiting passage of the biocompatible phase invertible composition. If so, evacuation of those fluids through the channel 17 may not be necessary. In all embodiments at least one fluid passage is provided to permit displaced fluid to escape from the mold space as the biocompatible phase invertible composition is injected into the mold space.

Once the proximal balloon 34 and distal balloon 36 are inflated as shown in FIG. 4c, fluid is injected into the mold space between the mandrel wall 38 and the lumen wall 64 in order to ensure that there is a seal. This can be achieved by increasing pressure on the bladder 21, by increasing pressure on fluid in tube 16c, or both. When the seal is verified, the biocompatible phase invertible composition is prepared and supplied to the tube 16c.

As explained above, the fluid in the micro-tube 16c, is forced through the nozzle 40, through the mold space 70, into the channel 17 (via the apertures 39 and fluid passages 31), and into the reservoir 19. The fluid in the tube 16c flushes the mold space of body fluids. FIG. 4d schematically represents this process at a point at which the initial fluid in the micro-tube 16c is removed and the biocompatible phase invertible composition has reached the mold space 70. At the opposite side of the mold space the initial fluid is being forced into the channel 17. Once the mold space 70 is filled with the biocompatible phase invertible composition, as shown in FIG. 4e, the composition is allowed to set. It will be appreciated by those skilled in the art that the supply of the biocompatible phase invertible composition and the rate of expansion of the bladder 21 are controlled to maintain a substantially constant pressure in the mold space, in order to maintain a constant volume of the mold space.

In accordance with the present embodiment, the biocompatible phase invertible composition sets within a predetermined time. In other embodiments, a curing agent, a change in temperature, pressure, etc. of the biocompatible phase invertible composition is used to trigger the setting of the biocompatible phase invertible composition, and the agent is applied in a suitable manner.

Once the biocompatible phase invertible composition in the mold space 70 has hardened sufficiently to provide a stent 72 that can structurally support the lumen, the proximal and distal balloons are deflated, retracting the mandrel wall 38, as shown in FIG. 4f for removal of the distal end unit 12. The proximal balloon 34, the distal balloon 36 and the mandrel wall 38 are coated with a lubricious surface treatment to facilitate separation from the stent. While the proximal balloon 34 and the distal balloon 36 are being deflated, fluid inside the mandrel is removed through the channel 17 to facilitate the collapse of the mandrel wall.

FIG. 4g shows the stent 72 in position, with the distal end unit 12 of the delivery system 10 removed.

Restenosis caused by smooth muscle proliferation subsequent to trauma is reduced using a stent in accordance with the present invention. In addition, the outer surface of the stent assumes the shape of the lumen. Thus, pressure points and pressure voids are eliminated. Trauma to the lumen wall is therefore further reduced, and this further reduces restenosis. In some embodiments and for certain procedures, a controlled release anti-restenosis agent may be mixed with the biocompatible phase invertible composition used to form the stent if desired. Short-term and long-term restenosis rates are predicted to be significantly reduced because of the reduced level of trauma required to mold the stent in situ, in comparison with prior art implanted stents.

Although the embodiments of the invention have been described above with reference to two balloons and a mandrel carried between the two balloons, it will be understood that the same function can accommodated with little modification using a single balloon having enlarged end sections and a middle section of a reduced diameter to provide the mandrel, so that the balloon is dumbbell shaped.

The embodiments of the invention described above are therefore intended to be exemplary only. The scope of the invention is therefore intended to be limited solely by the scope of the appended claims.

I Claim:

1. A method of molding a stent for supporting a lumen in a living body, the method comprising:
operating a stent delivery system to position a mandrel within the lumen at a site where the stent is to be molded;
operating the stent delivery system to define a mold space between the mandrel and the lumen; and
injecting a biocompatible phase invertible composition into the mold space to fill the mold space, the biocompatible phase invertible composition setting after a predetermined period of time to form a micro-porous stent that provides structural support for the lumen.

2. The method as claimed in claim 1 wherein operating the stent delivery system further comprises expanding distal and proximal balloons to define and seal off the mold space between the mandrel and a wall of the lumen.

3. The method as claimed in claim 1 wherein positioning the mandrel further comprises maneuvering a catheter of the stent delivery system through the lumen until the mandrel is positioned at the site.

4. The method as claimed in claim 2 wherein expanding the distal and proximal balloons seals the mold space and radially expands the mandrel.

5. The method as claimed in claim 3 wherein expanding the distal and proximal balloons comprises controlling a supply of a fluid within respective fluid conduits within the catheter.

6. The method as claimed in claim 1 wherein injecting the biocompatible phase invertible composition comprises controlling a supply of the biocompatible phase invertible composition into the mold space through a fluid conduit within the catheter that is in fluid communication with the mold space.

7. The method as claimed in claim 6 wherein injecting the biocompatible phase invertible composition further comprises waiting a predetermined period of time and withdrawing fluid from an interior of the mandrel to draw the mandrel away from the stent after the biocompatible phase invertible composition is set.

8. The method as claimed in claim 6 wherein injecting the biocompatible phase invertible composition further comprises:
controlling the supply of the biocompatible phase invertible composition into the fluid conduit of the catheter to introduce a pre-computed volume of the biocompatible phase invertible composition into the fluid conduit; and
controlling a supply of a chaser fluid into the fluid conduit to urge the biocompatible phase invertible composition through the fluid conduit and into the mold space.

9. The method as claimed in claim 8 further comprising waiting a predetermined time for the biocompatible phase invertible composition to set, and withdrawing at least a portion of a fluid from inside the mandrel to return the mandrel to a collapsed condition for withdrawal of the delivery system from the lumen.

10. The method as claimed in claim 8 wherein the chaser fluid comprises a biocompatible glycerol having a viscosity that is greater than a viscosity of the biocompatible phase invertible composition when it is injected into the mold space.

11. The method as claimed in claim 1 wherein the biocompatible phase invertible composition comprises a proteinaceous polymer.

12. The method as claimed in claim 11 wherein biocompatible phase invertible composition comprises comprises an aldehyde modified to be biocompatible, albumin and collagen.

13. The method as claimed in claim 11 wherein the biocompatible phase invertible composition is infused with an anti-restenosis agent.

14. A stent formed in situ within a living body comprising a biocompatible phase invertible composition molded to form the stent, the biocompatible phase invertible composition setting to form a rigid, micro-porous stent that provides structural support for a lumen in the living body.

15. The stent as claimed in claim 14 wherein the biocompatible phase invertible composition adhesively binds to a wall of the lumen.

16. The stent as claimed in claim 14 wherein the biocompatible phase invertible composition comprises a proteinaceous polymer.

17. The stent as claimed in claim 16 wherein biocompatible phase invertible composition comprises an aldehyde modified to be biocompatible, albumin and collagen.

18. The stent as claimed in claim 17 wherein the lumen is a blood vessel and the biocompatible phase invertible composition further comprises an anti-restenosis agent.

19. An apparatus for molding a stent at a selected site within a lumen of a living body, the apparatus comprising:
a catheter having a distal insertion end, and a proximal manipulation end;
a distal end unit on the distal insertion end of the catheter, the distal end unit being movable within the lumen by controlling the proximal manipulation end of the catheter;
a mandrel incorporated in the distal end unit, the mandrel being expandable from a collapsed insertion condition to an expanded molding condition in which a mold space is defined between a wall of the lumen and the mandrel; and
a conduit for injecting a biocompatible phase invertible composition into the mold space to fill the mold space, the biocompatible phase invertible composition providing a rigid micro-porous stent that provides structural support for the lumen after the biocompatible phase invertible composition has set.

20. The apparatus as claimed in claim 19 further comprising at least two balloons on the distal end unit adapted to be inflated to seal off the mold space and deflated for removal of the catheter from the living body.

21. The apparatus as claimed in claim 20 wherein the at least two balloons comprise a proximal and a distal balloon respectively located at opposite ends of the distal end unit, the proximal and distal balloons being inflatable to provide a fluid seal between the lumen and the mandrel at respective proximal and distal ends of the mandrel.

22. The apparatus as claimed in claim 21 wherein opposite ends of the mandrel are respectively tensionally connected to the proximal and distal balloons, so that inflation of the balloons radially expands the mandrel to the expanded molding condition to define the mold space.

23. The apparatus as claimed in claim 22 wherein the catheter comprises a multi-lumen catheter having a plurality of parallel fluid conduits that provide a plurality of isolated fluid communication paths between the manipulation end and the distal end unit.

24. The apparatus as claimed in claim 23 wherein a one of the fluid communication paths communicates with a nozzle secured to a wall of the mandrel to supply a biocompatible fluid to the mold space of the mandrel when the mandrel is in the expanded molding condition.

25. The apparatus as claimed in claim 24 wherein the mandrel wall includes at least one aperture on a side distant the nozzle, in order to permit fluid in the mold space to enter an interior of the mandrel for withdrawal through the one of the fluid communications paths so that the biocompatible fluid can displace the fluid in the mold space.

26. The apparatus as claimed in claim 24 wherein the one of the fluid communications paths is coupled to a pressurized flow controller for ensuring that a fluid pressure within the mold space is substantially constant as the biocompatible phase invertible composition is being injected into the mold space.

27. The apparatus as claimed in claim 23 further comprising an adapter of at least one of the fluid communications paths at the manipulation end of the multi-lumen catheter for coupling with a pressurized fluid controller for controlling a pressure of a fluid in the at least one of the fluid communications paths that is coupled to at least one of the proximal and distal balloons to inflate and deflate the coupled balloon.

28. The apparatus as claimed in claim 19 wherein the mandrel is provided with at least one fluid passage to permit displaced fluid to escape from the mold space as the biocompatible phase invertible composition is injected into the mold space.

29. The apparatus as claimed in claim 28 wherein the fluid passage to permit displaced fluid to escape is connected to a fluid reservoir.

30. The apparatus as claimed in claim 29 wherein the fluid reservoir comprises an elastic baldder.
